(19) United States
(12) Patent Application Publication
(10) Pub. No.: US 2024/0057878 A1
(43) Pub. Date: Feb. 22, 2024

(54) CONTINUOUS SELF-CALIBRATING BLOOD PRESSURE MONITORING DEVICE AND METHOD FOR ITS USE

(71) Applicant: TAIWAN AULISA MEDICAL DEVICES TECHNOLOGIES, INC., Taipei City (TW)

(72) Inventors: AUGUSTINE Y. LIEN, Taipei City (TW); CHI-HSIANG MA, Taipei City (TW); PO-MIN SHIH, Taipei City (TW)

(21) Appl. No.: 17/892,156

(22) Filed: Aug. 22, 2022

(51) Int. Cl. A61B 5/021; A61B 5/024

(52) U.S. Cl. CPC A61B 5/02108; A61B 5/02416

(57) ABSTRACT

The present invention provides devices and methods for self-calibrating and continuous blood pressure monitoring systems and for recording such measurements. The electrocardiography (ECG) signal and photoplethysmography (PPG) signal are measured in real-time then fit into a processing module, which generates a plurality of calibration parameters. A self-correcting pulse wave velocity and blood pressure relation equation, that is continuously and dynamically corrected by the plurality of calibration parameters, cooperates with the blood pressure state of the user to measure the blood pressure value of the user more accurately.

### BACKGROUND OF THE INVENTION

### 1. Field of the Invention

The present invention relates to devices and methods for self-calibrating and continuous blood pressure monitoring systems and for recording such measurements.

### 2. Description of the Related Art

Generally, a standard blood pressure meter pressurizes and decompresses the measurement site (upper arm or wrist) by utilizing a pressure cuff to indirectly obtain the blood pressure values by sensing the presence or absence of the pulse. However, the measurement method may significantly affect the measurement results. The error may be as the result of improper cuff placement. Additionally, if the same measurement site is repeatedly pressurized, it may cause elastic fatigue of the blood vessels at the measurement site, which in turn, may lead to measurement inaccuracies.

Other technologies utilizing wearable devices and optical sensing have also been proposed. Pulse Transit Time (PTT) is a physiological parameter that has been widely used for estimating Blood Pressure (BP). The usage of PTT can be dated back to 1964, when Weltman devised the Pulse Wave Velocity (PWV) computer by “utilizing the EKG complex and a downstream pulse signal to define pulse transit time over a known arterial length”. Based on this principle, the relationship between PWV and PTT is represented by the following equation (1).

$$P W V = \frac{( Distance between heart and fingertip , D )}{( Pulse Transit time )} \qquad ( 1 )$$

The relationship between PTT and PWV is used to relate blood pressure, and is represented by the following equation (2).

BP=a×PWV²+β (2)

Many prior arts proposed different equations similar to equation (2) for improving accuracy. However, such devices only use one formula to represent dynamic blood pressure. Thus, an extra blood pressure meter is often necessary to regularly correct the measurement results, which can cause inconvenience in measuring blood pressure.

### BRIEF SUMMARY OF THE INVENTION

In order to overcome the shortcomings of the above-mentioned prior art, the present invention provides devices and methods for continuous self-calibrating blood pressure measurement by recording vital signals in real-time. This invention combines three technologies: Pulse Wave Analysis technology, Fuzzy Decision Tree, and continuous non-invasive blood pressure measurement to achieve continuous self-calibration blood pressure measurement by the incoming user's physiology signals. In other words, this blood pressure measurement is further calibrated based on the continuous acquisition of users' ECG and PPG waveforms to enhance the accuracy of the estimated blood pressure.

The present invention provides a method for continuous self-calibrating blood pressure measurement. The method consists of the following steps: 1. obtain an electrocardiography (ECG) signal and a photoplethysmography (PPG) signal. 2. Input the ECG and the PPG signals into a processing module to calculate a plurality of calibration parameters. 3. Using the calibration weight value generation module within the processing module to calculate a calibration weight value. 4. Input the calibration weight value into a relation equation generation module within the processing module to compute a relation formula between pulse wave velocity and blood pressure. 5. Obtain the relation formula to the blood pressure information generation module within the processing module to calculate a self-calibrated blood pressure value in real-time by continuously physiology signals.

Among the calibration parameter generation module to analyze five features based on Pulse Wave Analysis, and the relation equation generation module includes a pre-trained equation which is the pulse wave velocity and the pulse transit time relationship related to blood pressure. FIG. 12, 134a demonstrates the pre-trained equation that uses blood pressure (BP) and pulse wave velocity (PWV) for human arteries via a two-parameter, quadratic formula: BP=a×PWV+b. Once a and b are determined, this formula could potentially be applied for cuff-less BP measurement. In some embodiments, one of the calibration parameters is associated with the change in the rate of slope change of PPG signal, defined as the ratio of a rising slope to a descending slope of the PPG signal.

Another objective of the present invention is to provide an electronic recording device. The device contains firmware to perform the following steps: obtains an ECG signal and a PPG signal; generates a plurality of calibration parameters by the obtained ECG and PPG signals; calculates a calibration weight value using the plurality of calibration parameters; self-calibrates an initial pulse wave velocity and blood pressure equation using the calibration weight value, obtains a self-calibrating pulse wave velocity and blood pressure equation; and obtains a self-calibrating blood pressure value by the self-correcting pulse wave velocity and blood pressure equation, wherein one of the plurality of calibration parameters is associated with a slope variation rate, and the slope variation rate is a ratio of a rising slope to a descending slope of the PPG signal.

Another objective of the present invention is to provide a continuous self-calibrating blood pressure measuring device comprising a plurality of electrocardiography signal sensing units, a photoplethysmography signal sensing unit and a processing module. The plurality of electrocardiography signal sensing units are used for sensing an initial electrocardiography signal. The photoplethysmography signal sensing unit is used for sensing an initial photoplethysmography signal. The processor module is electrically connected to the plurality of electrocardiography signal sensing units and the photoplethysmography signal sensing unit, and used for receiving the initial electrocardiography signal and the initial photoplethysmography signal. The processor module executes the following steps according to the initial electrocardiography signal and the initial photoplethysmography signal: obtains ECG signal and a PPG signal using the initial electrocardiography signal and the initial photoplethysmography signal; generates a plurality of calibration parameters by the obtained ECG signal and the PPG signal; calculates a calibration weight value by the plurality of calibration parameters; self-calibrates an initial pulse wave velocity and blood pressure equation using the calibration weight value, obtains a self-calibrating pulse wave velocity and blood pressure equation; and obtains a self-calibrating blood pressure value by the self-calibrating pulse wave velocity and blood pressure equation, wherein one of the plurality of calibration parameters is associated with a slope variation rate and the slope variation rate is a ratio of a rising slope to a descending slope of the PPG signal.

The above and other aspects of the invention will become better understood regarding the following detailed description of the preferred but non-limiting embodiments. The following description is made with reference to the accompanying drawings.

### BRIEF DESCRIPTION OF THE DRAWINGS

FIG. 1 is a schematic diagram illustrating a continuous self-calibrating blood pressure measurement device according to an embodiment of the present invention

FIG. 2 is a schematic diagram illustrating a host according to an embodiment of the present invention.

FIG. 3 is a schematic diagram illustrating a memory module to an embodiment of the present invention.

FIG. 4 is a schematic diagram illustrating a calibration parameter generation module to an embodiment of the present invention.

FIG. 5 is a schematic diagram illustrating a heart rate decision tree according to an embodiment of the present invention.

FIG. 6 is a schematic disclosure illustrating a calibration parameter table according to an embodiment of the present invention.

FIG. 7 is a schematic diagram illustrating a pulse transit time (PTT) decision tree according to an embodiment of the present invention.

FIG. 8 is a schematic diagram illustrating a photoplethysmography signal according to an embodiment of the present invention.

FIG. 9 is a schematic diagram illustrating a ratio of systolic index decision tree according to an embodiment of the present invention.

FIG. 10 is a schematic diagram illustrating a subendocardial viability ratio decision tree according to an embodiment of the present invention.

FIG. 11 is a schematic diagram illustrating a slope variation rate decision tree according to an embodiment of the present invention.

FIG. 12 is a schematic diagram illustrating an initial pulse wave velocity and blood pressure equation as well as a self-calibrating pulse wave velocity and blood pressure equation according to an embodiment of the present invention.

FIG. 13 is a schematic diagram illustrating steps of a blood pressure measurement method according to an embodiment of the present invention.

FIG. 14 is a schematic diagram illustrating steps of a blood pressure measurement method according to an embodiment of the present invention.

### DETAILED DESCRIPTION OF THE INVENTION

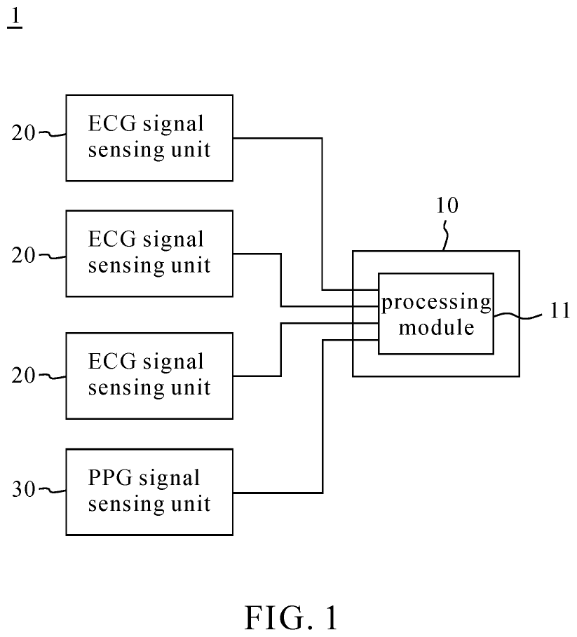

FIG. 1 is a schematic diagram illustrating a continuous self-calibrating blood pressure measurement device 1 according to an embodiment of the present invention. The continuous self-calibrating blood pressure measurement device 1 comprises a host 10, a plurality of electrocardiography (ECG) signal sensing units 20 and a photoplethysmography (PPG) signal sensing unit 30. The host 10 is electrically connected with the plurality of ECG signal sensing units 20 and the PPG signal sensing unit 30. Through physical signal wires, an initial ECG signal from the plurality of ECG signal sensing units 20, and an initial PPG signal from the PPG signal sensing unit 30 are received by processing module 11.

In the embodiment illustrated in FIG. 1, the plurality of ECG signal sensing units 20 may be implemented using sensing electrodes and the PPG signal sensing unit 30 may be implemented using an optical sensor.

The blood pressure measurement device 1 is used to measure physiological signals (i.e., the initial ECG signal and the initial PPG signal) of a user, in order to obtain the physiological state of the user.

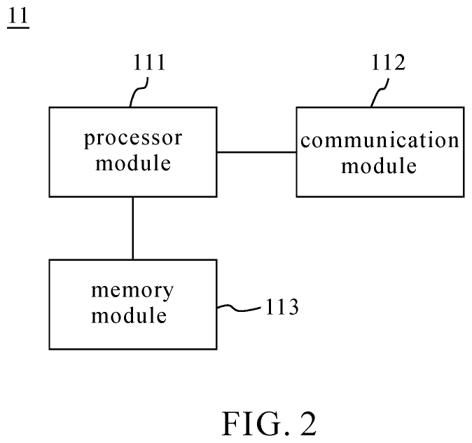

Referring now to FIG. 1 and FIG. 2, the host 10 includes at least a processing module 11. The processing module 11 includes at least a processor module 111, a communication module 112 and a memory module 113. The processor module 111 is electrically connected with the communication module 112 and the memory module 113, wherein the processor module 111 is used for executing a plurality of source codes stored in the memory module 113 (i.e., the electronic recording device), and is connected with an external device through the communication module 112.

In an embodiment, the processing module 11 may be implemented using a microcontroller.

In another embodiment, the processor module 111 may be implemented using a central processor.

In an embodiment, the communication module 112 may be implemented using a wireless communication circuit, for example, a Bluetooth communication module.

In another embodiment, the communication module 112 may be implemented using a wired communication circuit, for example, a Universal Serial Bus (USB) communication module.

In an embodiment, the memory module 113 may be implemented using flash memory, a memory card, or other non-volatile memory types which store a plurality of source codes.

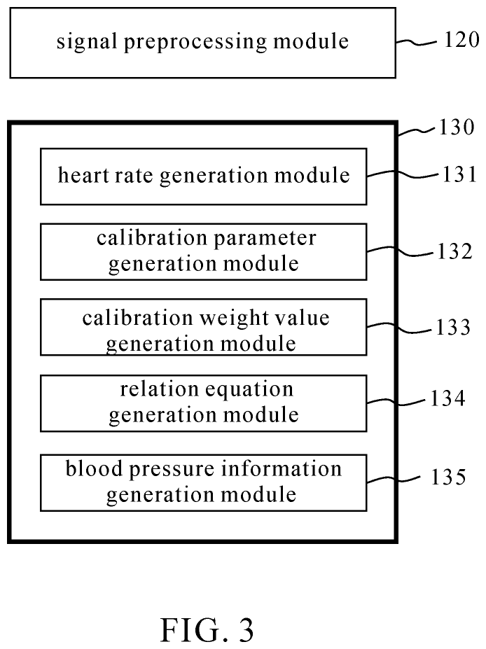

Referring to FIG. 2 and FIG. 3, in an embodiment, the memory module 113 consists of a signal preprocessing module 120 and a continuous self-calibrating blood pressure measurement module 130. In the embodiment, the signal preprocessing module 120 and the continuous self-calibrating blood pressure measurement module 130 may be implemented using a plurality of source codes.

The signal preprocessing module 120 is used for capturing and preprocessing the initial signal of ECG and PPG, e.g., amplification, polarity reversal to the initial ECG signal and the initial PPG signal, in order to generate an ECG signal and a PPG signal for calculating a self-calibrating blood pressure value.

The continuous self-calibrating blood pressure measurement module 130 further includes a heart rate generation module 131, a calibration parameter generation module 132, a calibration weight value generation module 133, a relation equation generation module 134 and a blood pressure information generation module 135.

The heart rate generation module 131 is used to calculate a heart rate (HR) based on the ECG signal. For example, the heart rate generation module 131 is used to determine the number of QRS waves included in the ECG signal within a fixed number of seconds, and accordingly calculates the number of heart beats per minute (HR), or the heart rate generation module 131 obtains a number of heart beats per minute according to a period D of the QRS waves.

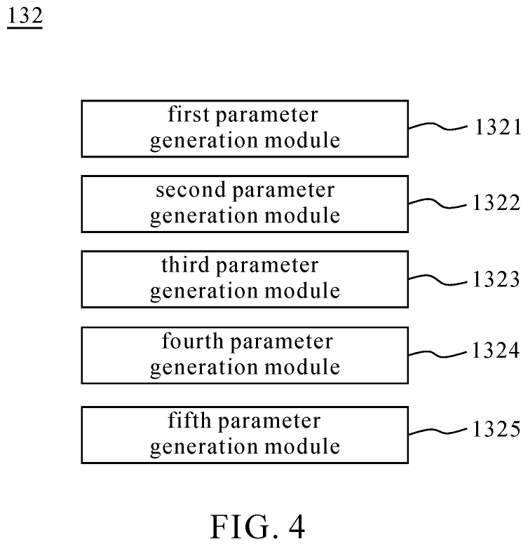

Based on a plurality of features from the ECG signal and the PPG signal, the calibration parameter generation module 132 is used to obtain a plurality of calibration parameters corresponding to these features. Further, referring to FIG. 4, the calibration parameter generation module 132 includes a first parameter generation module 1321, a second parameter generation module 1322, a third parameter generation module 1323, a fourth parameter generation module 1324 and a fifth parameter generation module 1325.

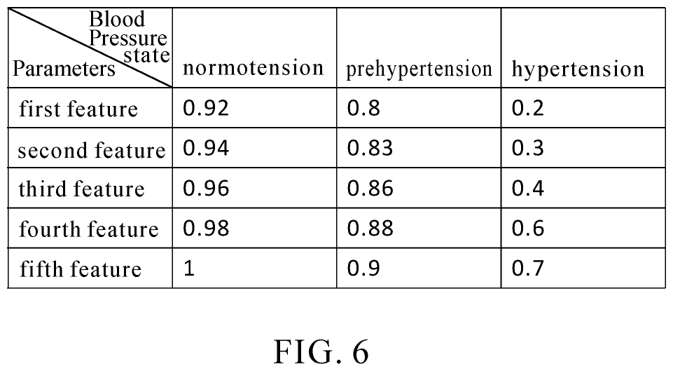

The first parameter generation module 1321 is used to obtain the first calibration parameter according to a first feature. Further, the first parameter generation module 1321 receives the heart rate (HR) (i.e., the first feature) calculated by the heart rate generation module 131. Then, the first parameter generation module 1321 determines the blood pressure state (normotension, prehypertension, hypertension) corresponding to the heart rate (HR) and obtains the first calibration parameter corresponding to the first feature in a calibration parameter table (FIG. 6) according to the judged blood pressure state.

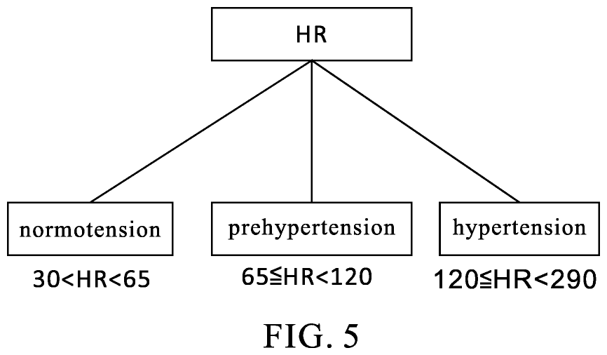

FIG. 5 is a schematic diagram illustrating a heart rate decision tree according to an embodiment of the present invention, where HR represents a heart rate (HR). After the first parameter generation module 1321 receives the heart rate (HR), the heart rate decision tree is used to determine the blood pressure state represented by that heart rate (HR). When the heart rate (HR) is greater than 30 bpm and less than 65 bpm, the state corresponding to the heart rate (HR) is normotension; {if 30<HR<65 then blood pressure state=normotension}. When the heart rate (HR) is greater than or equal to 65 bpm and less than 120 bpm, the state corresponding to the heart rate (HR) is prehypertension; {if 65≤HR≤120 then blood pressure state=prehypertension}. When the heart rate (HR)is greater than or equal to 120 bpm and less than 290 bpm, the state corresponding to the heart rate (HR) is hypertension; {if 120≤HR≤290 then blood pressure state=hypertension}. Next, the first parameter generation module 1321 looks up the first calibration parameter corresponding to the heart rate (HR) in the calibration parameter table (FIG. 6) according to the judged blood pressure state. For example, when the blood pressure state corresponding to the heart rate (HR) (i.e., the first feature) is normotension, the first calibration parameter is the value of 0.92 corresponding to the first feature and normotension in FIG. 6.

Among them, each correction parameter is defined as a Gaussian distribution value that each feature corresponds to a different blood pressure state as FIG. 6, that is, each correction parameter can be regarded as the probability (or correlation) of each feature in different blood pressure states.

Referring to FIG. 4 again, the second parameter generation module 1322 is used for obtaining the second calibration parameter according to a second feature. The second parameter generation module 1322 receives the ECG signal and the PPG signal, calculates the time lag between the R wave peak of the ECG signal and the P wave peak of the PPG signal, in order to obtain the pulse transit time (PTT) value as the second feature. The second parameter generation module 1322 also determines the corresponding blood pressure state according to the PTT value, and looks up and obtains the corresponding second calibration parameter in the calibration parameter table of FIG. 6 according to the judged blood pressure state.

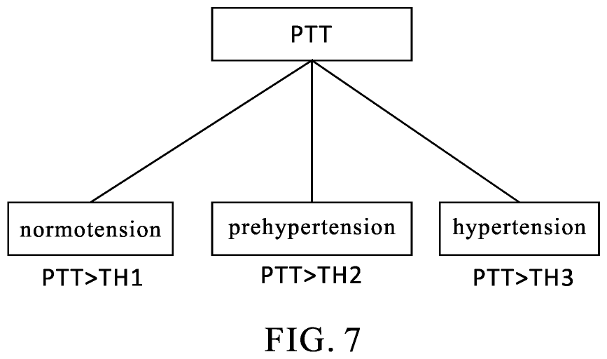

FIG. 7 is a schematic diagram of a PTT decision tree of an embodiment, wherein PTT represents a pulse transit time (PTT). After the second parameter generation module 1322 calculates a PTT value, the PTT decision tree is used to determine the blood pressure state represented by that PTT value. When the PTT value is greater than a first threshold value TH1, the state corresponding to the PTT value is normotension; {if TH1<PPT then blood pressure state=normotension}. When the PTT value is greater than a second threshold value TH2, the state corresponding to the PTT value is prehypertension; {if TH2<PPT then blood pressure state=prehypertension}. When the PTT value is greater than a third threshold value TH3, the state represented by the PTT value is hypertension; {if TH3<PPT then blood pressure state=hypertension}. The first threshold value TH1, the second threshold value TH2 and the third threshold value TH3 are obtained by the average value of the peak-to-peak value d of the P wave in the PPG signal. For example, the first threshold value TH1 is the average value of the peak-to-peak value d of the P wave multiplied by 0.5; the second threshold value TH2 is the average value of the peak-to-peak value d of the P wave multiplied by 0.6; and the third threshold value TH3 is the average value of the peak-to-peak value d of the P wave multiplied by 0.7. However, the present disclosure is not limited by the ratio. The second parameter generation module 1322 looks up the second calibration parameter corresponding to the PTT value in the calibration parameter table according to the blood pressure state represented by the PTT value. For example, when the state of the PTT value (i.e., the second feature) is normotension, the second correction parameter is the value of 0.94 corresponding to the second feature and normotension in FIG. 6.

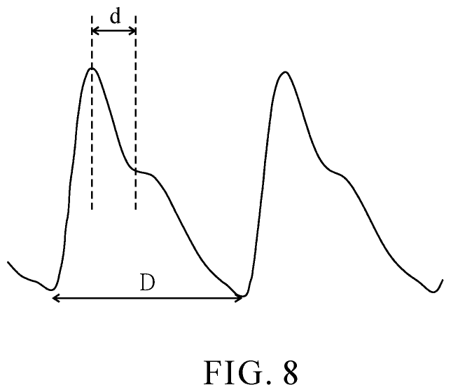

Referring to FIG. 8 and FIG. 4 again, FIG. 8 is a schematic diagram illustrating a PPG signal according to an embodiment of the present invention. The third parameter generation module 1323 is used for obtaining the third calibration parameter according to a third feature. Further, the third parameter generation module 1323 receives the PPG signal, and obtains a ratio systolic index (RSI) (the third feature) by a peak-to-peak value d of the P wave and the V (Valley) wave of the PPG signal (a time lag represented as d in FIG. 8) and a period D of the PPG signal (a time length represented as D in FIG. 8). The third parameter generation module 1323 determines the blood pressure state corresponding to the ratio systolic index (RSI), and looks up and obtains the corresponding third calibration parameter in the calibration parameter table according to the judged blood pressure state. In the embodiment illustrated in FIG. 4 and FIG. 8, the third parameter generation module 1323 uses the difference between the above-described peak-to-peak value d and the period D of the PPG signal (|d-D|) divided by the period D of the PPG signal to obtain the ratio systolic index (RSI). Therefore, the third feature reflects the conversion time of cardiac systole and diastole where the larger the value of the third feature is, the longer the conversion time is, the greater the vascular resistance is, and the more likely hypertension occurs.

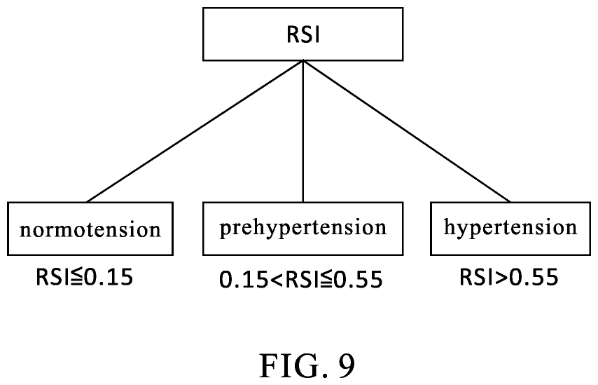

FIG. 9 is a schematic diagram illustrating a ratio systolic index (RSI) decision tree according to an embodiment of the ratio systolic index (RSI). After the third parameter generation module 1323 calculates a ratio systolic index (RSI), the ratio systolic index decision tree is used to determine the blood pressure state represented by that ratio systolic index (RSI). When the ratio systolic index (RSI) is less than or equal to 0.15, the state corresponding to the ratio systolic index (RSI) is normotension; {if RSI≤0.15 then blood pressure state=normotension}. When the ratio is greater than 0.15 and less than or equal to 0.55, the state corresponding to the ratio systolic index (RSI) is prehypertension; {if 0.15<RSI≤0.55 then blood pressure state=prehypertension}. When the ratio systolic index (RSI) is greater than 0.55, the state corresponding to the ratio is hypertension; {if RSI>0.55 then blood pressure state=hypertension}. Next, the third parameter generation module 1323 looks up and obtains the third calibration parameter corresponding to the ratio in the calibration parameter table (FIG. 6) according to the blood pressure state represented by the ratio systolic index (RSI). For example, when the blood pressure state of the ratio systolic index (RSI) (i.e., the third feature) is normotension, the third correction parameter is the value of 0.96 corresponding to the third feature and normotension in FIG. 6.

Referring to FIG. 4 again, the fourth parameter generation module 1324 is used for obtaining the fourth calibration parameter according to a fourth feature. Further, the fourth parameter generation module 1324 receives the PPG signal and calculates the subendocardial viability ratio (SEVR) (hereinafter referred to as SEVR) (i.e., the fourth feature) of the UP Stroke to the P wave peak of the PPG signal. The fourth parameter generation module 1324 then determines the blood pressure state corresponding to the SEVR; and looks up and obtains the corresponding fourth calibration parameter in the calibration parameter table according to the judged blood pressure state.

In the embodiment illustrated in FIG. 4, the fourth parameter generation module 1324 calculates the time lag from the UP Stroke to the P wave peak of the PPG signal, and divides the time lag by the average time of the peak-to-peak value d between the P waves of the PPG signal to obtain the SEVR. The lower the number of the SEVR is, the shorter the time of the UP Stroke to the peak of the P wave of the PPG signal is, i.e., the less the time required for the heart to output maximum blood, the better the SEVR is.

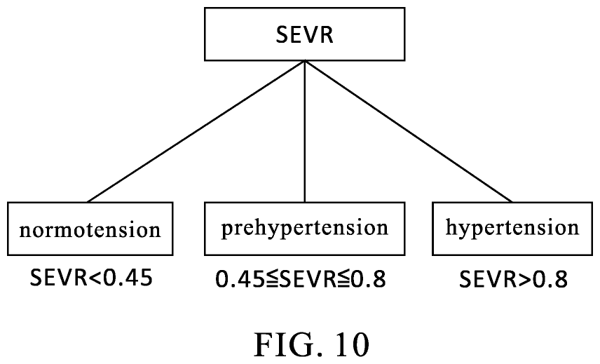

FIG. 10 is a schematic diagram illustrating a subendocardial viability ratio SEVR decision tree according to an embodiment of the present invention. After the fourth parameter generation module 1324 calculates a SEVR, the subendocardial viability ratio decision tree is used to determine the blood pressure corresponding to that SEVR. When the SEVR value is less than 0.45, the state represented by the SEVR is normotension; {if SEVR<0.45 then blood pressure state=normotension}. When the SEVR value is greater than or equal to 0.45 and less than or equal to 0.8, the state corresponding to the SEVR is prehypertension; {if 0.45≤SEVR≤0.8 then blood pressure state=prehypertension}. When the SEVR value is greater than 0.8, the state corresponding to the SEVR is hypertension; {if SEVR>0.8 then blood pressure state=hypertension}. Next, the fourth parameter generation module 1324 looks up the fourth calibration parameter corresponding to the SEVR in the calibration parameter table of FIG. 6 according to the blood pressure state corresponding to the SEVR. For example, when the state corresponding to the SEVR is normotension, the fourth calibration parameter is the value of 0.98 corresponding to the fourth feature and normotension as in FIG. 6.

Referring to FIG. 4 again, the fifth parameter generation module 1325 is used for obtaining the fifth calibration parameter according to a fifth feature. Further, the fifth parameter generation module 1325 calculates a rising slope Sr and a descending slope Sd of the PPG signal, and divides the rising slope Sr by the descending slope Sd to obtain a slope variation rate Sv (i.e., the fifth feature). The fifth parameter generation module 1325 then determines the blood pressure state corresponding to the slope variation rate Sv, and looks up and obtains the corresponding fifth calibration parameter in the calibration parameter table according to the judged blood pressure state.

In the embodiment illustrated in FIG. 4, the fifth parameter generation module 1325 is used for receiving the PPG signal, and calculates the rising slope Sr by the coordinates of the UP stroke and the coordinates of the P wave peak. The fifth parameter generation module 1325 also calculates the descending slope Sd by the coordinates of the P wave peak and the coordinates of the next UP stroke.

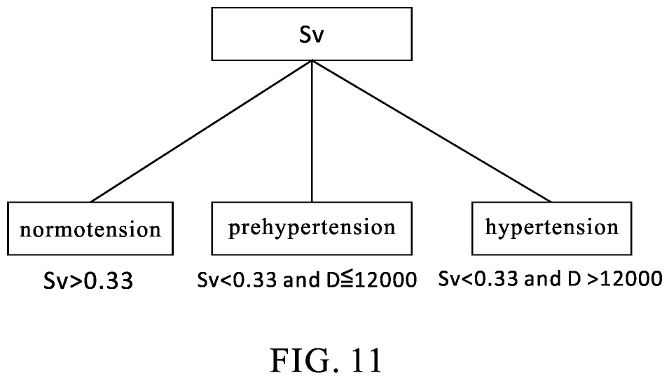

FIG. 11 is a schematic diagram illustrating a slope variation rate decision tree according to an embodiment of the present invention, where Sv represents the slope variation rate Sv. After the fifth parameter generation module 1325 receives a slope variation rate Sv, the slope variation rate decision tree is used to determine the blood pressure state represented by that slope variation rate Sv. When the slope variation rate Sv is greater than 0.33,the state represented by the slope variation rate Sv is normotension; {if Sᵥ>0.33 then blood pressure state=normotension}. When the slope variation rate Sv is less than 0.33 and the period D of the PPG signal (as represented by D shown in FIG. 8) is less than or equal to a threshold value (e.g., 12000), the state represented by the slope variation rate Sv is prehypertension; {if Sᵥ>0.33 &D≤12000 then blood pressure state=prehypertension}. When the slope variation rate Sv is less than 0.33 and the period D of the PPG signal (as D shown in FIG. 8) is greater than the threshold value (e.g., 12000), the state represented by the slope variation rate Sv is hypertension; {if S_v<0.33 & D>12000 then blood pressure state=hypertension}. Next, the fifth parameter generation module 1325 looks up the fifth calibration parameter corresponding to the slope variation in the calibration parameter table of FIG. 6 according to the blood pressure state represented by the slope variation rate Sv. For example, when the state of the slope variation (i.e., the fifth feature) is normotension, the fifth calibration parameter is the value of 1 corresponding to the fifth feature and normotension in FIG. 6.

The rising slope Sr, the descending slope Sd, and the length of the period D of the PPG signal are associated with the adaptive state of the blood vessel at the measurement site and the state of the blood pressure value. That is, the better the adaptability (expansion or contraction) of the blood vessel is, the higher the slope variation rate Sv of the PPG signal is, and the shorter the length of the period D is, the more normal the blood pressure value. Therefore, by the slope variation rate Sv, the state of the blood pressure value can be effectively discerned. In addition, as can be seen from FIG. 6, among the five features, the fifth feature has the highest correlation with blood pressure state, so the blood pressure of the user can be more accurately estimated by observing the fifth feature.

In the embodiment illustrated in FIG. 4 and FIG. 11, the heart rate decision tree, the PTT decision tree, the ratio systolic index decision tree, the subendocardial viability ratio decision tree and the slope variation rate decision tree are individually implemented by the fuzzy decision model. Each fuzzy decision model is based on actual clinical and physiological data, and the segmentation conditions are used to generate internal nodes. In the embodiment illustrated in FIG. 4 and FIG. 11, this may be implemented using Iterative Dichotomiser 3 (ID3), but the present invention is not limited thereto.

Referring to FIG. 3, the calibration weight value generation module 133 receives the above plurality of calibration parameters, and applies the plurality of calibration parameters to a calibration weight value equation to obtain a calibration weight value. The calibration weight value equation is as follows, with W representing the calibration weight value, P1 representing the first calibration parameter, P2 representing the second calibration parameter, P3 representing the third calibration parameter, P4 representing the fourth calibration parameter, and P5 representing the fifth calibration parameter:

W=0.2×P1+0.2×P2+0.2×P3+0.2×P4+0.2×P5

In the embodiment illustrated in FIG. 3, 0.2 is a variable parameter, the variable parameter represents the proportion of the effect of each feature on the blood pressure value. In the embodiment described by the above equation, for a general healthy adult, the five features have the same effect on the blood pressure value, so the variable parameter is 0.2.

In a situation when the slope variation rate Sv of FIG. 11 is less than 0.33 and the period D of the PPG signal is less than or equal to a threshold value, the calibration weight value equation is:

W=0.2×P1+0.2×P2+0.2×P3+0.1×P4+0.3×P5

In this example, since the slope variation rate Sv is less than 0.33 and the period D of the PPG signal is less than or equal to a threshold value, that represents the blood pressure value showing a prehypertensive state, the proportion of the fifth calibration parameter may directly reflect that the vascular adaptivity is increased to more accurately estimate the blood pressure value.

In a situation when the slope variation rate Sv of FIG. 11 is less than 0.33 and the period D of the PPG signal is greater than 12000, the calibration weight value equation is:

W=0.3×P1+0.2×P2+0.1×P3+0.1×P4+0.3×P5

In this example since the slope variation rate Sv is less than 0.33 and the period D of the PPG signal is greater than 12000 that represents the blood pressure value showing a hypertensive state. The first calibration parameter, positively related to blood pressure, and the proportion of the fifth correction parameter that may directly reflect the vascular adaptivity, is increased to estimate the blood pressure value more accurately.

Further, the relation equation generation module 134 self-corrects an initial pulse wave velocity and blood pressure relation equation 134a by the calibration weight value and obtains a self-calibrating pulse wave velocity and blood pressure relation equation 134b, wherein the initial pulse wave velocity and blood pressure relation equation 134a represents the relation between a pulse wave velocity (PWV) and blood pressure value by a linear equation. Further, the initial pulse wave velocity and blood pressure relation equation 134a is shown below, wherein X represents the pulse wave velocity, Y represents the blood pressure value, and a and b are variables. Variable a and variable b are based on a plurality of the relation between pulse transit time and pulse wave velocity related to blood pressure is used to establish the initial pulse wave velocity.

Y=aX+b

The relation equation generation module 134 corrects the initial pulse wave velocity and blood pressure relation equation 134a by the calibration weight value and obtains the self-calibrating pulse wave velocity and blood pressure relation equation 134b. The self-calibrating pulse wave velocity and blood pressure relation equation 134b is shown below, wherein X represents the pulse wave velocity, Y represents the blood pressure value, W represents the calibration weight value, and a and b are variables:

Y=aXW+bW

Thus, the self-calibrating pulse wave velocity and blood pressure relation equation 134b may continuously self-calibrate with the physiological signals from the user (ECG signal, PPG signal) by the calibration weight value, so that the linear relation between the pulse wave velocity and the blood pressure value is closer to the physiological state of the user, in order to more accurately measure the blood pressure value of the user.

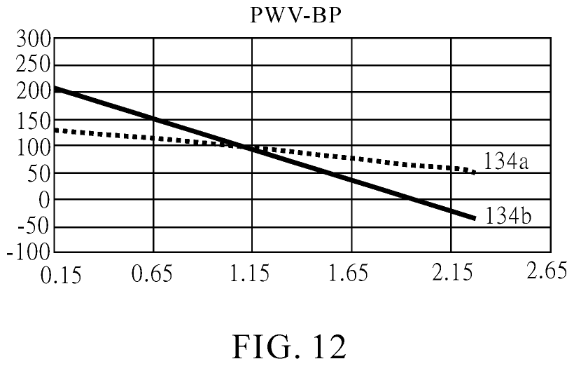

FIG. 12 is a schematic diagram illustrating an initial pulse wave velocity and blood pressure relation equation 134a as well as the self-calibrating pulse wave velocity and blood pressure relation equation 134b according to an embodiment of the present invention. In this embodiment, the initial pulse wave velocity and blood pressure relation equation 134a is Y=37.08X+136.12, and the self-calibrating pulse wave velocity and blood pressure relation equation 134b, after the adjustment of the calibration weight value, is Y=115.66X+225. Therefore, the relation between pulse wave velocity and blood pressure value is adjusted by the calibration weight value.

Referring to FIG. 3, the blood pressure information generation module 135 is used to obtain a self-calibrating blood pressure value by the self-calibrating pulse wave velocity and blood pressure relation equation 134b according to the received ECG signal and the PPG signal. Further, the blood pressure information generation module 135 calculates a corresponding pulse transit time (PTT) value from the ECG signal and the PPG signal, and obtains the corresponding PWV value according to the PWV=BCF×height/PTT. The PWV value (i.e., X) is brought into the self-calibrating pulse wave velocity and blood pressure relation equation 134b to calculate the self-calibrating blood pressure value (i.e., Y).

Thus, by the blood pressure measurement device 1 of the present invention, the blood pressure of the user may be measured continuously in a non-pressurized manner, based on the acquired physiological signal of the user (the ECG signal and the PPG signal). Additionally, the physiological signal of the user is used to immediately self-calibrate blood pressure estimation results, in order to accurately measure the blood pressure value of the user.

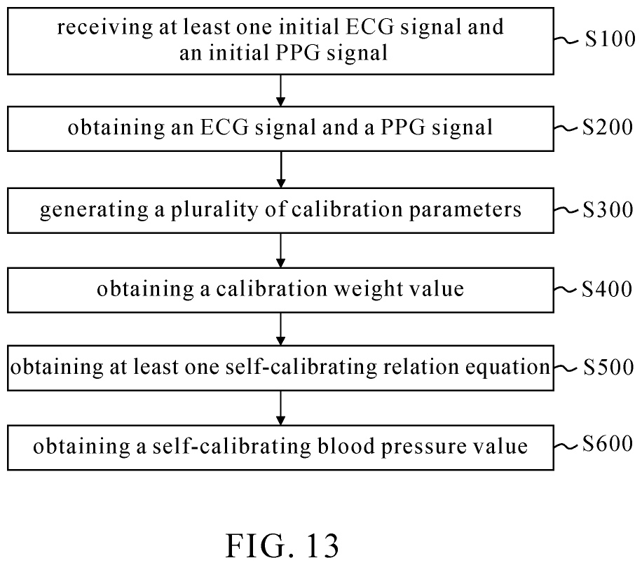

A blood pressure measurement method with continuous self-calibration may be summarized from the above embodiments of the present invention, and may be employed by the above blood pressure measurement device 1. The method comprises the following steps, as shown in FIG. 13.

Step S100: receiving at least one initial ECG signal and an initial PPG signal. A blood pressure measurement device 1 receives an initial ECG signal from a plurality of ECG signal sensing units 20 and an initial PPG signal from a PPG signal sensing unit 30 (as shown in FIG. 1).

Step S200: obtaining an ECG signal and a PPG signal. The blood pressure measurement device 1 comprises a signal preprocessing module 120. The signal preprocessing module 120 receives the initial ECG signal and the PPG signal, and carries out signal preprocessing for the initial ECG signal and the PPG signal to obtain the processed ECG signal and PPG signal (as shown in FIG. 3).

Step S300: generating a plurality of calibration parameters. The blood pressure measurement device 1 comprises a continuous self-calibrating blood pressure estimation module 130. The continuous self-calibrating blood pressure estimation module 130 is used for capturing a plurality of features in the ECG signal and the PPG signal, and looks up and obtains a plurality of calibration parameters in a calibration parameter table, according to the blood pressure state corresponding to each feature (as shown in FIG. 3). In the embodiment illustrated in FIG. 13, the plurality of calibration parameters includes a first calibration parameter, a second calibration parameter, a third calibration parameter, a fourth calibration parameter and a fifth calibration parameter.

Step S400: obtaining a calibration weight value. The continuous self-calibrating blood pressure estimation module 130 includes a calibration weight value generation module 133. The calibration weight value generation module 133 receives the above plurality of calibration parameters, and applies the plurality of calibration parameters to a calibration weight value equation to obtain a calibration weight value (as shown in FIG. 3).

In the embodiment illustrated in FIG. 13, the calibration weight value generation module 133 receives a first calibration parameter, a second calibration parameter, a third calibration parameter, a fourth calibration parameter and a fifth calibration parameter.

Step S500: obtaining at least one self-calibrating relation equation. The continuous self-calibrating blood pressure estimation module 130 includes a relation equation generation module 134. The relation equation generation module 134 self-corrects an initial pulse wave velocity and blood pressure relation equation 134a by the calibration weight value, and accordingly obtains the self-calibrating pulse wave velocity and blood pressure relation equation 134b. In the embodiment illustrated in

FIG. 13, the continuous self-calibrating blood pressure estimation module 130 makes the calibration weight value multiplied by the initial pulse wave velocity and blood pressure relation equation 134a to obtain the self-calibrating pulse wave velocity and blood pressure relation equation 134b after calibration.

Step S600: obtaining a self-calibrating blood pressure value. The continuous self-calibrating blood pressure estimation module 130 includes a blood pressure information generation module 135. The blood pressure information generation module 135 calculates a corresponding pulse transit time (PTT) value from the ECG signal and the PPG signal, and obtains the corresponding PWV value according to the conversion relation between the PTT value and the PWV value. The PWV value is brought into the self-calibrating pulse wave velocity and blood pressure relation equation 134b to calculate the self-calibrating blood pressure value.

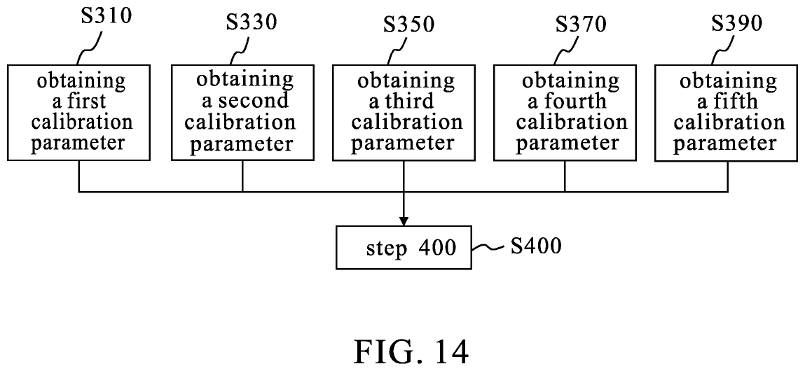

Step S300, further comprises the following steps, as shown in FIG. 14.

Step S310: obtaining a first calibration parameter. The continuous self-calibrating blood pressure estimation module 130 includes a heart rate generation module 131 (FIG. 3). The calibration parameter generation module 132 includes a first parameter generation module 1321 (FIG. 4). The first parameter generation module 1321 is used to obtain the first calibration parameter according to a first feature. Further, the first parameter generation module 1321 receives the heart rate (HR) (i.e., the first feature) calculated by the heart rate generation module 131, and determines the blood pressure state corresponding to the heart rate (HR) (normotension, prehypertension, hypertension) by a heart rate decision tree. The first parameter generation module 1321 looks up and obtains the first calibration parameter corresponding to the first feature in a calibration parameter table (FIG. 6) according to the judged blood pressure state. In an embodiment, when the heart rate (HR) is greater than 30 bpm and less than 65 bpm, the state corresponding to the heart rate (HR) is normotension. When the heart rate (HR) is greater than or equal to 65 bpm and less than 120 bpm, the state corresponding to the heart rate (HR) is prehypertension. When the heart rate (HR) is greater than or equal to 120 bpm and less than 290 bpm, the state corresponding to the heart rate (HR) is hypertension.

Step S330: obtaining a second calibration parameter. The calibration parameter generation module 132 includes a second parameter generation module 1322 (FIG. 4). The second parameter generation module 1322 is used for obtaining the second calibration parameter according to a second feature. Further, the second parameter generation module 1322 receives the ECG signal and the PPG signal. The second parameter generation module 1322 then calculates the time lag between the R wave peak of the ECG signal and the P wave peak of the PPG signal, in order to obtain a pulse transit time (PTT) value as the second feature. The second parameter generation module 1322 also determines the blood pressure state corresponding to the PTT value by a pulse transit time decision tree, and looks up and obtains the corresponding second calibration parameter in the calibration parameter table according to the judged blood pressure state.

In an embodiment, when the PTT value is greater than a first threshold value TH1, the state corresponding to the PTT value is normotension. When the PTT value is greater than a second threshold value TH2, the state corresponding to the PTT value is prehypertension. When the PTT value is greater than a third threshold value TH3, the state represented by the PTT value is hypertension. The first threshold value TH1, the second threshold value TH2 and the third threshold value TH3 are obtained by the average value of the peak-to-peak value d of the P wave in the PPG signal. For example, the first threshold value TH1 is the average value of the peak-to-peak value d of the P wave multiplied by 0.5. The second threshold value TH2 is the average value of the peak-to-peak value d of the P wave multiplied by 0.6. The third threshold value TH3 is the average value of the peak-to-peak value d of the P wave multiplied by 0.7. However, the present disclosure is not limited by these ratios.

Step S350: obtaining a third calibration parameter. The calibration parameter generation module 132 includes a third parameter generation module 1323 (FIG. 4). The third parameter generation module 1323 is used for obtaining a third calibration parameter according to a third feature. Further, the third parameter generation module 1323 receives the PPG signal, and obtains a ratio systolic index (RSI) (the third feature) by a period of peak-to-peak interval value of the P wave d and the V (Valley) wave of the PPG signal (a time lag represented as d in FIG. 8) and a period D of the PPG signal (a time length represented as D in FIG. 8). The third parameter generation module 1323 determines the blood pressure state corresponding to the ratio systolic index (RSI) by a ratio systolic index decision tree, and looks up and obtains the corresponding third calibration parameter in the calibration parameter table according to the judged blood pressure state. In an embodiment, the third parameter generation module 1323 calculates the difference between the above-described peak-to-peak value d and the period D of the PPG signal (|d-D|) divided by the period D of the PPG signal to obtain the ratio systolic index (RSI). In an embodiment, when the ratio systolic index (RSI) is less than or equal to 0.15, the state corresponding to the ratio systolic index (RSI) is normotension. When the ratio is greater than 0.15 and less than or equal to 0.55, the state corresponding to the ratio systolic index (RSI) is prehypertension. When the ratio systolic index (RSI) is greater than 0.55, the state corresponding to the ratio is hypertension.

Step S370: obtaining a fourth calibration parameter. The calibration parameter generation module 132 includes a fourth parameter generation module 1324 (FIG. 4). the fourth parameter generation module 1324 is used for obtaining a fourth calibration parameter according to a fourth feature. Further, the fourth parameter generation module 1324 receives the PPG signal, and calculates a subendocardial viability ratio (SEVR) (hereinafter referred to as SEVR) (i.e., the fourth feature) of the UP Stroke to the P wave peak of the PPG signal. The fourth parameter generation module 1324 determines the blood pressure state corresponding to the SEVR by a SEVR decision tree, and looks up and obtains the corresponding fourth calibration parameter in the calibration parameter table according to the judged blood pressure state. In an embodiment, when the SEVR value is less than 0.45, the state represented by the SEVR is normotension. When the SEVR value is greater than or equal to 0.45 and less than or equal to 0.8, the state corresponding to the SEVR is prehypertension. When the SEVR value is greater than 0.8, the state corresponding to the SEVR is hypertension.

Step S390: obtaining a fifth calibration parameter. The calibration parameter generation module 132 includes a fifth parameter generation module 1325 (FIG. 4). The fifth parameter generation module 1325 is used for obtaining a fifth calibration parameter according to a fifth feature. Further, the fifth parameter generation module 1325 calculates a rising slope Sr and a descending slope Sd of the PPG signal, and divides the rising slope Sr by the descending slope Sd to obtain a slope variation rate Sv (i.e., the fifth feature). The fifth parameter generation module 1325 determines the blood pressure state corresponding to the slope variation rate Sv by a slope variation rate decision tree, and looks up and obtains the corresponding fifth calibration parameter in the calibration parameter table according to the blood pressure state. In an embodiment, when the slope variation rate Sv is greater than 0.33, the state represented by the slope variation rate Sv is normotension. When the slope variation rate Sv is less than 0.33 and the period D of the PPG signal (as shown in FIG. 8) is less than or equal to 12000, the state represented by the slope variation rate Sv is prehypertension. When the slope variation rate Sv is less than 0.33 and the period D of the PPG signal (as shown in FIG. 8) is greater than 12000, the state represented by the slope variation rate Sv is hypertension.

After the above-described first calibration parameter to fifth calibration parameter are obtained, step S400 is performed successively.

In summary, by the blood pressure measurement device 1 with continuous self-calibration, the blood pressure measurement method with continuous self-calibration, and the electronic recording medium of the present invention, a plurality of calibration parameters associated with the blood pressure state can be obtained from a real-time measurement of an ECG signal and PPG signal. A self-correcting pulse wave velocity and blood pressure relation equation 134b, that is continuously and dynamically corrected by the plurality of calibration parameters, cooperates with the blood pressure state of the user to more accurately measure the blood pressure value of the user.

What is claimed is:

1. A continuous self-calibrating blood pressure measurement method, comprising:
obtaining an ECG signal and a PPG signal;
generating a plurality of calibration parameters via the ECG signal and the PPG signal;
obtaining a calibration weight value by the plurality of calibration parameters;
self-calibrating an initial pulse wave velocity and blood pressure relation equation by the calibration weight value, and obtaining a self-calibrating pulse wave velocity and blood pressure relation equation; and
obtaining a self-calibrating blood pressure value by the self-calibrating pulse wave velocity and blood pressure relation equation,
wherein one of the plurality of calibration parameters is associated with a slope variation rate, the slope variation rate is a ratio of a rising slope to a descending slope of the PPG signal.

2. The blood pressure measurement method according to claim 1, wherein the step of generating a plurality of calibration parameters comprises:
generating a plurality of features, the plurality of features include the slope variation rate;
determining the blood pressure state corresponding to each the plurality of features; and
looking up and obtaining the calibration parameter corresponding to each the plurality of features in a calibration parameter table according to the judged blood pressure state.

3. The blood pressure measurement method according to claim 2, wherein when the slope variation rate is greater than 0.33, the blood pressure state is normotension;
when the slope variation rate is less than 0.33 and a period of a PPG signal is less than or equal to a threshold value, the blood pressure state is prehypertension; and
when the slope variation rate is less than 0.33 and the period of the PPG signal is greater than the threshold value, the blood pressure state is hypertension.

4. The blood pressure measurement method according to claim 1, wherein the plurality of calibration parameters are individually multiplied by a variable parameter and then accumulated by each other to obtain the calibration weight value.

5. The blood pressure measurement method according to claim 1, wherein the calibration weight value is multiplied by the initial pulse wave velocity and blood pressure relation equation to obtain the self-calibrating pulse wave velocity and blood pressure relation equation.

6. An electronic device readable recording medium, storing a plurality of source codes to make an electronic device carry out the following steps upon executing the plurality of source codes:
obtaining an ECG signal and a PPG signal;
generating a plurality of calibration parameters by the ECG signal and the PPG signal;
calculating a calibration weight value by the plurality of calibration parameters;
self-calibrating an initial pulse wave velocity and blood pressure relation equation by the calibration weight value, and obtaining a self-calibrating pulse wave velocity and blood pressure relation equation; and
obtaining a self-calibrating blood pressure value by the self-calibrating pulse wave velocity and blood pressure relation equation, wherein one of the plurality of calibration parameters is associated with a slope variation rate, the slope variation rate is a ratio of a rising slope to a descending slope of the PPG signal.

7. The electronic device readable recording medium according to claim 6, wherein the step of generating a plurality of calibration parameters comprises:
generating a plurality of features, the plurality of features include the slope variation rate;
determining the blood pressure state corresponding to each the plurality of features; and
looking up and obtaining the calibration parameter corresponding to each the plurality of features in a calibration parameter table according to the judged blood pressure state.

8. The electronic device readable recording medium according to claim 7, wherein when the slope variation rate is greater than 0.33, the blood pressure state is normotension; when the slope variation rate is less than 0.33 and a period of a PPG signal is less than or equal to a threshold value, the blood pressure state is prehypertension; and when the slope variation rate is less than 0.33 and the period of the PPG signal is greater than the threshold value, the blood pressure state is hypertension.

9. The electronic device readable recording medium according to claim 6, wherein the plurality of calibration parameters are individually multiplied by a variable parameter and then accumulated by each other to obtain the calibration weight value.

10. The electronic device readable recording medium according to claim 6, wherein the calibration weight value is multiplied by the initial pulse wave velocity and blood pressure relation equation to obtain the self-calibrating pulse wave velocity and blood pressure relation equation.

11. A continuous self-calibrating blood pressure measurement device comprising:
a plurality of electrocardiography signal sensing units, used for sensing and outputting an initial electrocardiography signal;
a photoplethysmography signal sensing unit, used for sensing and outputting an initial photoplethysmography signal; and
a processing module, electrically connected with the plurality of electrocardiography signal sensing units and the photoplethysmography signal sensing unit for receiving the initial electrocardiography signal and the initial photoplethysmography signal, and executing the following steps:
obtaining an ECG signal and a PPG signal using the initial electrocardiography signal and the initial photoplethysmography signal;
generating a plurality of calibration parameters using the ECG signal and the PPG signal;
calculating a calibration weight value using the plurality of calibration parameters;
self-calibrating an initial pulse wave velocity and blood pressure relation equation by the calibration weight value, and obtaining a self-calibrating pulse wave velocity and blood pressure relation equation; and
obtaining a self-calibrating blood pressure value using the self-calibrating pulse wave velocity and blood pressure relation equation,
wherein one of the plurality of calibration parameters is associated with a slope variation rate, the slope variation rate is a ratio of a rising slope to a descending slope of the PPG signal.

12. The blood pressure measurement device according to claim 11, wherein the step of the processing module generating a plurality of calibration parameters comprises:
generating a plurality of features, the plurality of features include the slope variation rate;
determining the blood pressure state corresponding to each the plurality of features; and
looking up and obtaining the calibration parameter corresponding to each of the plurality of features in a calibration parameter table according to the judged blood pressure state.

13. The blood pressure measurement device according to claim 12, wherein when the slope variation rate is greater than 0.33: the state represents normal normotension;
when the slope variation rate is less than 0.33 and a period of a PPG signal is less than or equal to a threshold value: the state represents prehypertension; when the slope variation rate is less than 0.33 and the period of the PPG signal is greater than the threshold value: the state represents hypertension.

14. The blood pressure measurement device according to claim 11, wherein the processing module makes the plurality of calibration parameters individually multiplied by a variable parameter and then accumulated by each other to obtain the calibration weight value.

15. The blood pressure measurement device according to claim 11, wherein the processing module utilizes the calibration weight value multiplied by the initial pulse wave velocity and blood pressure relation equation in order to obtain the self-calibrating pulse wave velocity and blood pressure relation equation.
